SEQUENCE LISTING (31 sequences)

SEQ ID NO: 1 - protein, 57 aa, synthetic construct
  UNSURE 1: note = X is C,P,G,A,V,I,M,L,F,Y,W,S,T,N,Q,D,E,R,K,or H
  UNSURE 2: note = X is P,G,A,V,I,M,L,Y,S,T,N,Q,D,E,R,K, or H
  UNSURE 3: note = X is C,P,G,A,V,I,M,L,F,Y,W,S,T,N,Q,D,E,R,K, or H
  UNSURE 4: note = X is C,G,V,I,M,L,F,D,E,R, or K
  UNSURE 5: note = X is V or L
  UNSURE 6: note = X is G,A,S,T,R,K, or H
  UNSURE 7: note = X is C,G,A,V,I,M,L,F,Y,W,S,T,N,Q,D,E,R,K, or H
  UNSURE 8: note = X is M,L, or R
  UNSURE 9: note = X is G,A,V,I,T, or K
  UNSURE 10: note = X is C,G,A,V,I,M,L,F,Y,W,S,T,N,Q,D,E,R,K, or H
  UNSURE 11: note = X is C,G,A,V,I,M,L,F,Y,W,S,T,N,Q,D,E,R,K, or H
  UNSURE 12: note = X is L,Y,R, or K
  UNSURE 13: note = X is V,Y,W,S,T, or N
  UNSURE 14: note = X is C,G,A,V,I,M,L,F,Y,W,S,T,N,Q,D,E,R,K, or H
  UNSURE 15: note = X is C,G,A,V,I,M,L,F,Y,W,S,T,N,Q,D,E,R,K, or H
  UNSURE 16: note = X is C,A,M,L,F,Y,W,N,Q,D,R, or K
  UNSURE 17: note = X is C,G,A,V,M,L,F,Y,W,S,T,N,Q,D,E,R,K, or H
  UNSURE 18: note = X is L or F
  UNSURE 19: note = X is P,G,A,V,I,M,L,F,Y,S,T,N,Q,E,R,K, or H
  UNSURE 20: note = X is C,P,G,A,V,I,M,L,F,Y,W,S,T,N,Q,D,E,R,K, or H
  UNSURE 22: note = X is P,G,A,F,Y,S,E, or H
  UNSURE 23: note = X is C,A,S,T,N,Q,E,R,K, or H
  UNSURE 24: note = X is R or K
  UNSURE 25: note = X is P, M or T
  UNSURE 27: note = X is P,G,A,V,F,Y,S,T,N,Q,D,E,R,K, or H
  UNSURE 28: note = X is P,V,S,T,N,Q,R,K, or H
  UNSURE 29: note = X is A,F, or H
  UNSURE 30: note = X is I,M,L, or T
  UNSURE 31: note = X is S,R,K, or H
  UNSURE 32: note = X is A,V,I,S,T,R,K, or H
  UNSURE 33: note = X is A or S
  UNSURE 34: note = X is M,L,W,S,T,Q, or R
  UNSURE 35: note = X is G,A,V,I,M,L,F,Y,W,S,T,N,Q,D,E,R,K, or H
  UNSURE 36: note = X is M,L,Q, or E
  UNSURE 37: note = X is G or H
  UNSURE 38: note = X is V,L,F, or Y
  UNSURE 39: note = X is C,P,G,A,V,I,M,L,S,T,N,Q,D,E,R,K, or H
  UNSURE 40: note = X is C,G,A,V,F,Y,S,T,N,Q,E,R,K, or H
  UNSURE 41: note = X is C,P,G,A,V,I,M,L,F,Y,W,S,T,N,Q,D,E,R,K, or H
  UNSURE 42: note = X is Q,R, or K
  UNSURE 43: note = X is I or S
  UNSURE 44: note = X is A,V,M,W,S,T,Q,E,R,K, or H
  UNSURE 45: note = X is C,M,S,N,Q, or D
  UNSURE 46: note = X is G,A, or N
  UNSURE 47: note = X is V,I, or L
  UNSURE 48: note = X is C,G,A,V,I,M,L,F,Y,W,S,N,Q,D,E,R,K, or H
  UNSURE 50: note = X is V or L
  UNSURE 51: note = X is A or S
  UNSURE 52: note = X is C,G,A,V,I,M,L,F,Y,W,S,T,N,Q,D,E,R, or H
  UNSURE 54: note = X is A or D
  UNSURE 55: note = X is G,M,F,Y,W,R, or H
  UNSURE 56: note = X is C,P,G,A,V,I,M,L,F,Y,W,S,T,N,Q,D,E,R,K, or H
  UNSURE 57: note = X is I,F,Y,N,K, or H
XXXXXXXXXXXXXXXXXXXXRXXXXLXXXXXXXXXXXXXXXXXXXXXXQXXXQXXXX

SEQ ID NO: 2 - protein, 57 aa, synthetic construct
  UNSURE 2: note = X is A, D or E
  UNSURE 5: note = X is P,G,A,V,I,M,L,F,Y,W,S,T,N,Q,E,R,K, or H
  UNSURE 6: note = X is C,A,V,I,M,L,F,Y,W,S,T,N,Q,D,E,R,K, or H
  UNSURE 7: note = X is V or I
  UNSURE 8: note = X is P,N, or H
  UNSURE 9: note = X is M,Y,W,S,T,N,D,E,R, or H
  UNSURE 10: note = X is I,L,F, or Y
  UNSURE 11: note = X is M,L, or F
  UNSURE 12: note = X is V,I,M,F, or Y
  UNSURE 13: note = X is G,A,S,N,Q,R,K, or H
  UNSURE 14: note = X is I or S
  UNSURE 15: note = X is N,Q, or H
  UNSURE 16: note = X is G,A,M,F,Y,S,T,N,Q,D,E,R,K, or H
  UNSURE 17: note = X is A,M,L,F,Y,N,Q,R,K, or H
  UNSURE 18: note = X is C,A,V,S,T, or E
  UNSURE 19: note = X is V,I,M,F,Y,W, or H
  UNSURE 20: note = X is R,K, or H
  UNSURE 21: note = X is G,R,K, or H
  UNSURE 22: note = X is A,M,L,F,Y,W,S,N,Q,E,R,K, or H
  UNSURE 23: note = X is P,G,A,V,S,T,Q,R, or H
  UNSURE 24: note = X is P,G,A,V,I,M,L,F,Y,W,S,T,N,Q,D,E,R,K, or H
  UNSURE 25: note = X is P,G,A,M,S,Q,D,E,R,K, or H
  UNSURE 26: note = X is A or D
  UNSURE 27: note = X is F,Y,W,T,R, or H
  UNSURE 28: note = X is C,A,I,M,L,F,Y,S,N,Q,D,E,R,K, or H
  UNSURE 29: note = X is C,P,G,A,V,I,M,L,F,Y,W,S,T,N,Q,E,R,K, or H
  UNSURE 30: note = X is L or F
  UNSURE 31: note = X is C,G,A,V,I,M,L,F,Y,W,S,T,N,Q,D,E,R,K, or H
  UNSURE 32: note = X is G,A,V,I,M,Y,S,T,N,Q,D,E,R,K, or H
  UNSURE 33: note = X is M,L,W, or R
  UNSURE 34: note = X is A,V,I,S,T,N,Q,D,E,R, or K
  UNSURE 35: note = X is C,G,A,V,I,M,L,F,Y,W,S,T,N,Q,D,E,R,K, or H
  UNSURE 36: note = X is I,M, or L
  UNSURE 37: note = X is P,A,S,E,R,K, or H
  UNSURE 38: note = X is C,G,A,V,I,M,L,F,Y,W,S,T,N,Q,D,E,R,K, or H
  UNSURE 39: note = X is A,V,I,M,L,F,Y,W,T,N,Q,E,R,K, or H
  UNSURE 40: note = X is F,Y,W,N,K,H, or T
  UNSURE 41: note = X is G,M,N,Q,D,R,K, or H
  UNSURE 42: note = X is A,S,T,Q,D,E,K,H, or N
  UNSURE 45: note = X is M,L,F, or Y
  UNSURE 46: note = X is G,A,V,I,F,Y,W,Q,E,R,K, or H
  UNSURE 47: note = X is V,I, or N
  UNSURE 49: note = X is C,V,L,F,Y,W,T, or H
  UNSURE 50: note = X is G or E
  UNSURE 52: note = X is M,L,F, or D
  UNSURE 53: note = X is M,L,S,T,N,Q,D,E, or R
  UNSURE 54: note = X is G,S,N,R, or H
  UNSURE 57: note = X is G,A,V,M,L,W,S,T,N,Q,E,R,K, or H
DXVQXXXXXXXXXXXXXXXXXXXXXXXXXXXXXXXXXXXXXXPSXXXAXXAXXXVIX

SEQ ID NO: 3 - protein, 56 aa, synthetic construct
  UNSURE 1: note = X is P or D
  UNSURE 2: note = X is C,G,V,L,S,N,Q,D,E,R,K, or H
  UNSURE 3: note = X is G,A,V,M,W,D,E,R, or K
  UNSURE 4: note = X is G,V,I,M,L, or T
  UNSURE 5: note = X is C,Y,W,T,N,D,R,K, or H
  UNSURE 6: note = X is I,M,T,N,R,K, or H
  UNSURE 7: note = X is C,V,M, or F
  UNSURE 8: note = X is V,M,F, or W
  UNSURE 9: note = X is A,I,M,Y,W,T,N,Q,E,K, or H
  UNSURE 10: note = X is G,I,M,L,Y,R,K, or H
  UNSURE 11: note = X is G,I,L, or W
  UNSURE 12: note = X is C,G,A,S,N,Q,E,R,K, or H
  UNSURE 13: note = X is C,G,A,V,I,M,L,Y,W,N,Q,D,E,R,K, or H
  UNSURE 14: note = X is A,V,Y, or F
  UNSURE 15: note = X is A,M,F,Y,W,S, or N
  UNSURE 16: note = X is C,G,V,I,M,F,Y,S,N,E,R,K, or H
  UNSURE 17: note = X is C,A,I,M,L,W,S,N,Q,R,K, or H
  UNSURE 18: note = X is A,L,T, or R
  UNSURE 19: note = X is G,A,V,I,M,L,Y,W,N,Q,D,E,R, or K
  UNSURE 20: note = X is G,A,V,M,F,S,T,Q,E,R,K, or H
  UNSURE 21: note = X is P,G,A,M,Y,E, or R
  UNSURE 22: note = X is C,I,M,L,F,W,S,T,N,Q,D,E,K, or H
  UNSURE 23: note = X is P,G,M,L,F,N,R,K, or H
  UNSURE 24: note = X is C,P,G,A,I,L,F,W,S,T,N,Q,D,E,K, or H
  UNSURE 25: note = X is A,M,F,S,N,R,K, or H
  UNSURE 26: note = X is G,A,V,F,S,T,N,R, or H
  UNSURE 27: note = X is C,G,A,V,I,M,L,F,Y,W,S,T,N,Q, or R
  UNSURE 28: note = X is C,V,I,F,Y,W,S,N,Q,D,E, or H
  UNSURE 29: note = X is C,A,V, or K
  UNSURE 31: note = X is A,I,M,W,S,T,N,D,E, or R
  UNSURE 32: note = X is G,A,V,I,L,S,D,E, or R
  UNSURE 34: note = X is C,G,A,V,L,F,Y,S,N,Q,E,R, or H
  UNSURE 35: note = X is G,A,I,M,L,F,W,T,Q,D,E, or R
  UNSURE 36: note = X is C,V,L,F,W,Q,D,R,K, or H
  UNSURE 38: note = X is C,G,V,I,F,Q,R,K,or H
  UNSURE 39: note = X is C,A,M,Y,W,N,Q,D,E, or R
  UNSURE 40: note = X is A,L,F,N, or H
  UNSURE 41: note = X is G,A,M,L,F,S,T,N,Q,D,R, or K
  UNSURE 42: note = X is Q or D
  UNSURE 43: note = X is C,P,G,A,I,M,L,F,W,S,T,N,E,R,K, or H
  UNSURE 44: note = X is G,F,S, or T
  UNSURE 46: note = X is A,I,T,E, or R
  UNSURE 47: note = X is A,R, or S
  UNSURE 48: note = X is V or F
  UNSURE 49: note = X is A or V
  UNSURE 50: note = X is I,L,F,T,N,Q,D, or H
  UNSURE 51: note = X is A,V,I,L,W,T, or D
  UNSURE 52: note = X is V,I,T, or H
  UNSURE 53: note = X is A,I,M,L,F,Y,W,S,T,N,Q,D,E,R,K, or H
  UNSURE 54: note = X is P,T,N,Q, or R
  UNSURE 55: note = X is P,V,L,W, or N
  UNSURE 56: note = X is A,L,Y,W,R, or K
XXXXXXXXXXXXXXXXXXXXXXXXXXXXXLXXAXXXAXXXXXXXLXXXXXXXXXXX

SEQ ID NO: 4 - protein, 57 aa, synthetic construct
DEERLRELVEELVKELGLSERAKRMLEQFLRSALEQGFDEEQIRMALEQLAEQAREI

SEQ ID NO: 5 - protein, 57 aa, synthetic construct
DEERLRELVEELVKELGLSERYKRTLERHLRTALEQGYDEEQIRMNLEQLAQQAREI

SEQ ID NO: 6 - protein, 59 aa, synthetic construct
CGDEERLRELVEELVKELGLSERYKRTLERHLRTALEQGYDEEQIRMNLEQLAQQAREI

SEQ ID NO: 7 - protein, 57 aa, synthetic construct
DEERLRCLVEELVKELGLSERYKRTLERHLRTALEQGYDEEQIRMNLEQLAQQAREI

SEQ ID NO: 8 - protein, 57 aa, synthetic construct
DEERLRELVCELVKELGLSERYKRTLERHLRTALEQGYDEEQIRMNLEQLAQQAREI

SEQ ID NO: 9 - protein, 57 aa, synthetic construct
DEERLRELVECLVKELGLSERYKRTLERHLRTALEQGYDEEQIRMNLEQLAQQAREI

SEQ ID NO: 10 - protein, 57 aa, synthetic construct
DEERLRELVEELVKELGLSERYKRTLCRHLRTALEQGYDEEQIRMNLEQLAQQAREI

SEQ ID NO: 11 - protein, 57 aa, synthetic construct
DEERLRELVEELVKELGLSERYKRTLERHLRTALEQGYDEEQIRMNLCQLAQQAREI

SEQ ID NO: 12 - protein, 57 aa, synthetic construct; identical to SEQ ID NO: 5

SEQ ID NO: 13 - protein, 57 aa, synthetic construct
SEEELKEEVEELVKKLGNKERYKRTLERHLRTALEQGYSEEQIRMNLEQLAQQAKKI

SEQ ID NO: 14 - protein, 57 aa, synthetic construct
SEEELEEEVEELVKELGNKERYKRTLERHLETALEQGYSEEQIRMNLEQLAQQAKKI

SEQ ID NO: 15 - protein, 57 aa, synthetic construct
DLETLRETVERLTAEIDGDERYRRTLERHADTAVKQGYDEETIEMNLRQLAEQLRLI

SEQ ID NO: 16 - protein, 57 aa, synthetic construct
SSEEVKELLERLKEEIGEDERYARTLERHYETAKKQGYDDETIAMNLKQLAEQAKLI

SEQ ID NO: 17 - protein, 57 aa, synthetic construct
AEERLWELVEELKKELGLCERHKKTLDRHLRTALEQGYDEEQIYMNLEQLAWQAKEI

SEQ ID NO: 18 - protein, 120 aa, synthetic construct
DEERLRELVEELVKELGLSERYKRTLERHLRTALEQGYDEEQIRMNLEQLAQQAREIGGSGGSDEERLRELVEELVKELGLSERYKRTLERHLRTALEQGYDEEQIRMNLEQLAQQAREI

SEQ ID NO: 19 - protein, 57 aa, synthetic construct
DEVQHEVHELFVRIQELVVRGNPEEARKLLEELEELAKKYNDPSLHIAVEALERVIN

SEQ ID NO: 20 - protein, 57 aa, synthetic construct
DEVQHEVHELMVRIHSLVVRGNPEEARKLLEELEELAKKTNNPSYHIAVEALEHVIN

SEQ ID NO: 21 - protein, 59 aa, synthetic construct
CGDEVQHEVHELMVRIHSLVVRGNPEEARKLLEELEELAKKTNNPSYHIAVEALEHVIN

SEQ ID NO: 22 - protein, 56 aa, synthetic construct
DEDTRRVMELLHEASELREEGDPERAREVLEEAERLAKELGDPSLERFVQAHKRVY

SEQ ID NO: 23 - protein, 56 aa, synthetic construct
DEDTDRVFELLNEFNELREEGDPERAREVLEEAERLAKELSDPTLESFVQAIKRVY

SEQ ID NO: 24 - protein, 58 aa, synthetic construct
CGDEDTDRVFELLNEFNELREEGDPERAREVLEEAERLAKELSDPTLESFVQAIKRVY

SEQ ID NO: 25 - protein, 56 aa, synthetic construct
DEDTDRVFECLNEFNELREEGDPERAREVLEEAERLAKELSDPTLESFVQAIKRVY

SEQ ID NO: 26 - protein, 56 aa, synthetic construct
DEDTDRVFELLNCFNELREEGDPERAREVLEEAERLAKELSDPTLESFVQAIKRVY

SEQ ID NO: 27 - protein, 56 aa, synthetic construct
DEDTDRVFELLNEFNELREEGDPERARECLEEAERLAKELSDPTLESFVQAIKRVY

SEQ ID NO: 28 - protein, 56 aa, synthetic construct
DEDTDRVFELLNEFNELREEGDPERAREVLCEAERLAKELSDPTLESFVQAIKRVY

SEQ ID NO: 29 - protein, 56 aa, synthetic construct
DEDTDRVFELLNEFNELREEGDPERAREVLEEAERLAKCLSDPTLESFVQAIKRVY

SEQ ID NO: 30 - protein, 118 aa, synthetic construct
DEDTDRVFELLNEFNELREEGDPERAREVLEEAERLAKELSDPTLESFVQAIKRVYGGSGGSDEDTDRVFELLNEFNELREEGDPERAREVLEEAERLAKELSDPTLESFVQAIKRVY

SEQ ID NO: 31 - protein, 6 aa, synthetic construct
GGSGGS

(19) United States
(12) Patent Application Publication
(10) Pub. No.: US 2025/0250301 A1
(43) Pub. Date: Aug. 7, 2025

(54) DE NOVO DESIGNED BINDERS TARGETING HUMAN EPCAM AND PDL1 RECEPTORS

(71) Applicant: University of Washington, Seattle, WA (US)

(72) Inventors: David BAKER,, Seattle, WA (US); Brian COVENTRY, Seattle, WA (US); Inna GORESHNIK, Seattle, WA (US); Derrick HICKS, Seattle, WA (US); Jilliane Ruth PERKINS, Seattle, WA (US); Daniel Adriano SILVA MANZANO, Seattle, WA (US); Samer HALABIYA, Seattle, WA (US); Vanessa PENG, Seattle, WA (US); Jue WANG, Seattle, WA (US)

(21) Appl. No.: 18/856,829

(22) PCT Filed: Apr. 27, 2023
(86) PCT No.: PCT/US2023/066279

§ 371 Date: Oct. 14, 2024

Related U.S. Application Data
(60) Provisional application No. 63/336,629, filed on Apr. 29, 2022.

(51) Int. Cl. C07K 14/00; A61K 38/00

(52) U.S. Cl. CPC C07K 14/001; A61K 38/00; C07K 2319/00

(57) ABSTRACT

Disclosed herein are polypeptides that bind to the epithelial cell adhesion molecule (EpCAM) and programmed death-ligand I (PDL 1) receptors, nucleic acids encoding them, and methods for their use in treating or limited development of cancer, auto immune disease, or inflammation. The disclosure further provides nucleic acids encoding a polypeptide of the disclosure: expression vector comprising a nucleic acid of the disclosure operatively linker to a suitable regulatory control element, host cells comprising a polypeptide, fusion protein, nucleic acid, or expression vector of any embodiment of the disclosure, and pharmaceutical compositions, comprising the polypeptide, fusion protein, nucleic acid, expression vector, or host cell of any embodiment; and a pharmaceutically acceptable carrier.

### CROSS REFERENCE

This application claims priority to U.S. Provisional Patent Application Ser. No. 63/336,629 filed Apr. 29, 2022, incorporated by reference herein in its entirety.

### FEDERAL FUNDING STATEMENT

This invention was made with government support under Grant No. HR0011835403 awarded by the Defense Advanced Research Projects Agency, and Grant No. RO1 CA240339-01 awarded by the National Cancer Institute, and Grant No. T32GM008268 awarded by the National Institute of General Medical Sciences, and Grant No. 5U19AG065156-02 awarded by the National Institute on Aging. The government has certain rights in the invention.

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the XML file created on Apr. 19, 2023 having the name “22-0544-WO.xml” and is 87,939 bytes in size.

### BACKGROUND

Human epithelial cell adhesion molecule (EpCAM) and programmed death-ligand 1 (PDL1) receptors are implicated in a range of diseases such as cancer, autoimmunity and inflammation. Thus, therapeutics targeting EpCAM and PDL1 are needed. While antibodies exist targeting these receptors, our designed proteins have unique properties compared to antibodies, such as rapid pharmacokinetics and enhanced tissue/tumor penetration due to their small size which would change the toxicity/efficacy profile of therapeutics made from our binders compared to antibodies.

### SUMMARY

In one aspect, the disclosure provides polypeptides that bind to human epithelial cell adhesion molecule (EpCAM) receptor, wherein the polypeptide comprises the amino acid sequence of SEQ ID NO:1. In one embodiment, interface residues 21, 22, 24, 25, 28, 29, 32, 33, 36, 42, 45, 46, 49, 50, 52, 53, 54, and 57 are selected from the corresponding interface residues present in any one of SEQ ID NO:4-18. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:4-18.

In another aspect, the disclosure provides polypeptides that binds to human EpCAM receptor, wherein the polypeptide comprises the amino acid sequence of SEQ ID NO:2. In one embodiment, interface residues 1, 4, 5, 7, 8, 9, 11, 12, 15, 16, 19, 20, 43, 44, 45, 47, 48, 50, 51, 52, 54, and 55 are selected from the interface residues present in the amino acid sequence selected from SEQ ID NO:19-21. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:19-21.

In a further aspect, the disclosure provides polypeptides that bind to human programmed death-ligand 1 (PDL1) receptor, wherein the polypeptide comprises the amino acid sequence of SEQ ID NO:3. In one embodiment, interface residues 1, 4, 5, 8, 11, 12, 15, 16, 19, 42, 43, 44, 47, 48, 51, 52, 54, 55, and 56 are selected from the interface residues present in the amino acid sequences selected from SEQ ID NO:22-30. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:22-30.

In another embodiment, the disclosure provides fusion proteins 2, 3, 4, or more polypeptides according to any embodiment of the disclosure, wherein the polypeptides may be directly linked, or may be linked via an amino acid linker.

The disclosure further provides nucleic acids encoding a polypeptide of the disclosure; expression vector comprising a nucleic acid of the disclosure operatively linker to a suitable regulatory control element, host cells comprising a polypeptide, fusion protein, nucleic acid, or expression vector of any embodiment of the disclosure, and pharmaceutical compositions, comprising the polypeptide, fusion protein, nucleic acid, expression vector, or host cell of any embodiment; and a pharmaceutically acceptable carrier.

The disclosure also provides methods for treating or limited development of cancer, autoimmune disease, or inflammation, comprising administering to a subject in need thereof an amount effective of the polypeptide, fusion protein, nucleic acid, expression vector, host cell, or pharmaceutical composition of any embodiment herein to treat or limit development of the disorder.

### DESCRIPTION OF THE FIGURES

FIG. 1. Binder design models from left to right: EpcamBind1_cb4, EpcamBind2_cb10, and PDL1Bind_cb6.

FIG. 2A. Octet data for EpcamBind1_cb4. EpcamBind1 shows association and dissociation curves and has an estimated Kd of 5.6 nM based on kinetic fitting to association and dissociation. Concentrations span a range from 100 nM (top curve) to 1.56 nM (bottom curve) by two-fold dilution.

FIG. 2B. Octet data for EpcamBind2_cb10. EpcamBind2 shows association and dissociation curves and has an estimated Kd of 500 nM based on kinetic fitting to association and dissociation. Concentrations span a range from 1000 nM (top curve) to 15.6 nM (bottom curve) by two-fold dilution.

FIG. 2C. Octet data for PDL1Bind_cb6. PDL1Bind_cb6 has an estimated Kd of 2 nM based on steady state analysis.

FIG. 3. Cell binding assay. Binding of control antibodies are shown on the top and the binding for the designed binders are shown the bottom. EpCAMBind1_cterm-cys-AF594 specifically binds to K562 cells engineered to express high levels of EpCAM without noticeable nonspecific binding to EpCAM-knockout K562 cells. Cell binding activity was observed at 50 nM and 500 nM. PDL1Bind1_E39C-AF594 specifically binds to PDL1+SKOV3 cells at 500 nM and 5 uM with some minor nonspecific binding to PDL1− cells at 5 uM, comparable to the nonspecific binding seen from the control antibody.

FIG. 4. Octet data for gs linked dimer of EpCAMBind1_cterm_cys. The plot shows association and dissociation curves and the binder has an estimated Kd of <10 pM based on kinetic fitting to association and dissociation. Concentrations from top curve to bottom curve are 20 nM, 10 nM, 5 nM, 2.5 nM, 1 nM and 0.5 nM.

FIG. 5. Sequence and secondary structure diagram for designs EpcamBind1_cb4 (SEQ ID NO: 5), EpcamBind2_cb10 (SEQ ID NO: 20), and, PDL1Bind_cb6 (SEQ ID NO: 23). Alpha helices are shown as boxes, loops are shown as lines, and interface residues are labeled with an asterisk.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press). Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be deleted).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides that binds to human epithelial cell adhesion molecule (EpCAM) receptor, wherein the polypeptide comprises the amino acid sequence of SEQ ID NO: 1.

As disclosed in the examples, the polypeptides of this aspect bind to the human EpCAM receptor. As disclosed in the examples that follow, the inventors have designed polypeptides according to SEQ ID NO:1 (exemplary such polypeptides are listed in Table 2), and conducted site saturation mutagenesis studies to determine permissible substitutions at each position, reflecting the allowable amino acids shown in Table 1. In some embodiments, these polypeptides bind to the extracellular portion of human EpCAM receptor.

Table 1 shows the amino acid sequence of SEQ ID NO:1

TABLE 1
SEQ ID NO: 1

| Residue number | Allowable amino acids |
|---|---|
| 1 | C, P, G, A, V, I, M, L, F, Y, W, S, T, N, Q, D, E, R, |
| | K, H |
| 2 | P, G, A, V, I, M, L, Y, S, T, N, Q, D, E, R, K, H |
| 3 | C, P, G, A, V, I, M, L, F, Y, W, S, T, N, Q, D, E, R, |
| | K, H |
| 4 | C, G, V, I, M, L, F, D, E, R, K, |
| 5 | V, L |
| 6 | G, A, S, T, R, K, H |
| 7 | C, G, A, V, I, M, L, F, Y, W, S, T, N, Q, D, E, R, K, |
| | H |
| 8 | M, L, R |
| 9 | G, A, V, I, T, K |
| 10 | C, G, A, V, I, M, L, F, Y, W, S, T, N, Q, D, E, R, K, |
| | H |
| 11 | C, G, A, V, I, M, L, F, Y, W, S, T, N, Q, D, E, R, K, |
| | H |
| 12 | L, Y, R, K |
| 13 | V, Y, W, S, T, N |
| 14 | C, G, A, V, I, M, L, F, Y, W, S, T, N, Q, D, E, R, K, |
| | H |
| 15 | C, G, A, V, I, M, L, F, Y, W, S, T, N, Q, D, E, R, K, |
| | H |
| 16 | C, A, M, L, F, Y, W, N, Q, D, R, K |
| 17 | C, G, A, V, M, L, F, Y, W, S, T, N, Q, D, E, R, K, H |
| 18 | L, F |
| 19 | P, G, A, V, I, M, L, F, Y, S, T, N, Q, E, R, K, H |
| 20 | C, P, G, A, V, I, M, L, F, Y, W, S, T, N, Q, D, E, R, |
| | K, H |
| Interface 21 | R |
| Interface 22 | P, G, A, F, Y, S, E, H |
| 23 | C, A, S, T, N, Q, E, R, K, H |
| Interface 24 | R, K |
| Interface 25 | P, M, T |
| 26 | L |
| 27 | P, G, A, V, F, Y, S, T, N, Q, D, E, R, K, H |
| Interface 28 | P, V, S, T, N, Q, R, K, H |
| Interface 29 | A, F, H |
| 30 | I, M, L, T |
| 31 | S, R, K, H |
| Interface 32 | A, V, I, S, T, R, K, H |
| Interface 33 | A, S |
| 34 | M, L, W, S, T, Q, R |
| 35 | G, A, V, I, M, L, F, Y, W, S, T, N, Q, D, E, R, K, H |
| Interface 36 | M, L, Q, E |
| 37 | G, H |
| 38 | V, L, F, Y |
| 39 | C, P, G, A, V, I, M, L, S, T, N, Q, D, E, R, K, H |
| 40 | C, G, A, V, F, Y, S, T, N, Q, E, R, K, H |
| 41 | C, P, G, A, V, I, M, L, F, Y, W, S, T, N, Q, D, E, R, |
| | K, H |
| Interface 42 | Q, R, K |
| 43 | I, S |
| 44 | A, V, M, W, S, T, Q, E, R, K, H |
| Interface 45 | C, M, S, N, Q, D |
| Interface 46 | G, A, N |
| 47 | V, I, L |
| 48 | C, G, A, V, I, M, L, F, Y, W, S, N, Q, D, E, R, K, H |
| Interface 49 | Q |
| Interface 50 | V, L |
| 51 | A, S |
| Interface 52 | C, G, A, V, I, M, L, F, Y, W, S, T, N, Q, D, E, R, H |
| Interface 53 | Q |
| Interface 54 | A, D |
| 55 | G, M, F, Y, W, R, H |
| 56 | C, P, G, A, V, I, M, L, F, Y, W, S, T, N, Q, D, E, R, |
| | K, H |
| Interface 57 | I, F, Y, N, K, H |

Table 1 also shows the position of interface residues in SEQ ID NO:1, at residues 21, 22, 24, 25, 28, 29, 32, 33, 36, 42, 45, 46, 49, 50, 52, 53, 54, and 57. In one embodiment, interface residues 21, 22, 24, 25, 28, 29, 32, 33, 36, 42, 45, 46, 49, 50, 52, 53, 54, and 57 are selected from the corresponding interface residues present in any one of SEQ ID NO:4-18 (sequences shown in Table 2).

The residue number of interface residues are listed in SEQ ID NO:1/Table 1, and the residue numbers of SEQ ID NO:4-18 in Table 2 are the same as those listed in SEQ ID NO:1/Table 1, except for:
SEQ ID NO:6 (EpCAM1_nterm-cys): This embodiment has two additional amino acid residues at the N-terminus relative to SEQ ID NO:1 *i.e., numbering shifted +2); and
SEQ ID NO:18 (EpCAM1_dimer): This embodiment includes two copies of the EpCAM receptor binder. The first copy follows the residue numbering in SEQ ID NO:1/Table 1. The second copy of the sequence (i.e.: residue 1 of the second copy) begins after the lower case “ggsggs” (SEQ ID NO: 31) linker sequence.

For context, bold-font residues in SEQ ID NO:5 are interface residues 21, 22, 24, 25, 28, 29, 32, 33, 36, 42, 45, 46, 49, 50, 52, 53, 54, and 57.

TABLE 2

| SEQ | | |
|---|---|---|
| ID | | |
| NO | Design | Sequence |
| 4 | EpcamBind1 | DEERLRELVEELVKELGLSERAKRMLEQF |
| | | LRSALEQGEDEEQIRMALEQLAEQAREI |
| 5 | EpcamBind1_ | DEERLRELVEELVKELGLSERYKRTLERH |
| | cb4 | LRTALEQGYDEEQIRMNLEQLAQQAREI |
| 6 | EpCAMI_ | CgDEERLRELVEELVKELGLSERYKRTLE |
| | nterm-cys | RHIRTALEQGYDEEQIRMNLEQLAQQARE |
| | | I |
| 7 | EpCAM1_E7C | DEERLRcLVEELVKELGLSERYKRTLERH |
| | | LRTALEQGYDEEQIRMNLEQLAQQAREI |
| 8 | EpCAM1_E10C | DEERLRELVcELVKELGLSERYKRTLERH |
| | | LRTALEQGYDEEQIRMNLEQLAQQAREI |
| 9 | EpCAM1_E11C | DEERLRELVEcLVKELGLSERYKRTLERH |
| | | ELRTALEQGYDEQIRMNLEQLAQQAREI |
| 10 | EpCAM1_E27C | DEERLRELVEELVKELGLSERYKRTLCRH |
| | | IRTALEQGYDEEQIRMNLEQLAQQAREI |
| 11 | EpCAM1_E48C | DEERLRELVEELVKELGLSERYKRTLERH |
| | | LRTALEQGYDEEQIRMNICQLAQQAREI |
| 12 | EpcamBind1_ | DEERIRELVEELVKELGLSERYKRTLERH |
| | cb4_cys | IRTALEQGYDEEQIRMNLEQLAQQAREI |
| 13 | EpcamBind1_ | SEEELKEEVEELVKKLGNKERYKRTLERH |
| | cb4-mod1 | LRTALEQGYSEEQIRMNLEQLAQQAKKI |
| 14 | EpcamBind1_ | SEEELEEEVEELVKELGNKERYKRTLERH |
| | cb4-mod2 | LETALEQGYSEEQIRMNLEQLAQQAKKI |
| 15 | EpcamBind1_ | DLETLRETVERLTAEIDGDERYRRTLERH |
| | cb4-mod3 | ADTAVKQGYDEETIEMNLRQLAEQLRLI |
| 16 | EpcamBind1_ | SSEEVKELLERLKEEIGEDERYARTLERH |
| | cb4-mod4 | YETAKKQGYDDETIAMNLKQLAEQAKLI |
| 17 | EpcamBind1_ | AEERLWELVEELKKELGLCERHKKTLDRH |
| | cb4-mod5 | LRTALEQGYDEEQIYMNLEQLAWQAKEI |
| 18 | EpCAM1_ | DEERLRELVEELVKELGLSERYKRTLERH |
| | dimer | LRTALEQGYDEEQIRMNLEQLAQQAREIg |
| | | gsggsDEERLRELVEELVKELGLSERYKR |
| | | TLERHLRTALEQGYDEEQIRMNLEQLAQQ |
| | | AREI |

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:4-18. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5 (EpcamBind1_cb4). In one such embodiment, interface residues in the polypeptide are identical to the reference polypeptide sequence. For example, residues 21, 22, 24, 25, 28, 29, 32, 33, 36, 42, 45, 46, 49, 50, 52, 53, 54, and 57 of the polypeptide (or corresponding residues in SEQ ID NO:6 or 18) are identical to the reference polypeptide sequence.

In another embodiment, residues 1-16 relative to the reference sequence (or corresponding residues in SEQ ID NO:6 or 18) form a first alpha-helix, residues, residues 20-36 relative to the reference sequence (or corresponding residues in SEQ ID NO:6 or 18) form a second alpha-helix, and residues 40-57 relative to the reference sequence form a third alpha-helix (or corresponding residues in SEQ ID NO:6 or 18). Residues present in alpha-helices are underlined in Table 2 for context. In a further embodiment, residues present in alpha-helices (“alpha-helical residues”) are identical to the reference sequence alpha-helical residues.

In another aspect, the disclosure provides polypeptides that binds to human EpCAM receptor, wherein the polypeptide comprises the amino acid sequence of SEQ ID NO:2.

As disclosed in the examples that follow, the inventors have designed polypeptides according to SEQ ID NO:2 (exemplary such polypeptides are listed in Table 4), and conducted site saturation mutagenesis studies to determine permissible substitutions at each position, reflecting the allowable amino acids shown in Table 3. In some embodiments, these polypeptides bind to the extracellular portion of human EpCAM receptor

TABLE 3
SEQ ID NO: 2

| Residue number | Allowable amino acids |
|---|---|
| 1 Interface | D |
| 2 | A, D, E |
| 3 | V |
| 4 Interface | Q |
| 5 Interface | P, G, A, V, I, M, L, F, Y, W, S, T, N, Q, E, R, K, H |
| 6 | C, A, V, I, M, L, F, Y, W, S, T, N, Q, D, E, R, K, H |
| 7 Interface | V, I |
| 8 Interface | P, N, H |
| 9 Interface | M, Y, W, S, T, N, D, E, R, H |
| 10 | I, L, F, Y |
| 11 Interface | M, L, F |
| 12 Interface | V, I, M, F, Y |
| 13 | G, A, S, N, Q, R, K, H |
| 14 | I, S |
| 15 Interface | N, Q, H |
| 16 Interface | G, A, M, F, Y, S, T, N, Q, D, E, R, K, H |
| 17 | A, M, L, F, Y, N, Q, R, K, H |
| 18 | C, A, V, S, T, E |
| 19 Interface | V, I, M, F, Y, W, H |
| 20 Interface | R, K, H |
| 21 | G, R, K, H |
| 22 | A, M, L, F, Y, W, S, N, Q, E, R, K, H |
| 23 | P, G, A, V, S, T, Q, R, H |
| 24 | P, G, A, V, I, M, L, F, Y, W, S, T, N, Q, D, E, R, K, |
| | H |
| 25 | P, G, A, M, S, Q, D, E, R, K, H |
| 26 | A, D |
| 27 | F, Y, W, T, R, H |
| 28 | C, A, I, M, L, F, Y, S, N, Q, D, E, R, K, H |
| 29 | C, P, G, A, V, I, M, L, F, Y, W, S, T, N, Q, E, R, K, |
| | H |
| 30 | L, F |
| 31 | C, G, A, V, I, M, L, F, Y, W, S, T, N, Q, D, E, R, K, |
| | H |
| 32 | G, A, V, I, M, Y, S, T, N, Q, D, E, R, K, H |
| 33 | M, L, W, R |
| 34 | A, V, I, S, T, N, Q, D, E, R, K |
| 35 | C, G, A, V, I, M, L, F, Y, W, S, T, N, Q, D, E, R, K, |
| | H |
| 36 | I, M, L |
| 37 | P, A, S, E, R, K, H |
| 38 | C, G, A, V, I, M, L, F, Y, W, S, T, N, Q, D, E, R, K, |
| | H |
| 39 | A, V, I, M, L, F, Y, W, T, N, Q, E, R, K, H |
| 40 | F, Y, W, N, K, H, T |
| 41 | G, M, N, Q, D, R, K, H |
| 42 | A, S, T, Q, D, E, K, H, N |
| 43 Interface | P |
| 44 Interface | S |
| 45 Interface | M, L, F, Y |
| 46 | G, A, V, I, F, Y, W, Q, E, R, K, H |
| 47 Interface | V, I, N |
| 48 Interface | A |
| 49 | C, V, L, F, Y, W, T, H |
| 50 Interface | G, E |
| 51 Interface | A |
| 52 Interface | M, L, F, D |
| 53 | M, L, S, T, N, Q, D, E, R |
| 54 Interface | G, S, N, R, H |
| 55 Interface | V |
| 56 | I |
| 57 | G, A, V, M, L, W, S, T, N, Q, E, R, K, H |

Table 3 also shows the position of interface residues in SEQ ID NO:2, at residues 1, 4, 5, 7, 8, 9, 11, 12, 15, 16, 19, 20, 43, 44, 45, 47, 48, 50, 51, 52, 54, and 55. In one embodiment, interface residues 1, 4, 5, 7, 8, 9, 11, 12, 15, 16, 19, 20, 43, 44, 45, 47, 48, 50, 51, 52, 54, and 55 are selected from the corresponding interface residues present in any one of SEQ ID NO:19-21 (sequences shown in Table 2).

The residue number of interface residues are listed in SEQ ID NO:2/Table 3, and the residue numbers of SEQ ID NO:19-21 in Table 4 are the same as those listed in SEQ ID NO:2/Table 3, except for SEQ ID NO:21 (EpcamBind2_cb10_cys). This embodiment has two additional amino acid residues at the N-terminus relative to SEQ ID NO:2 *i.e., numbering shifted +2).

TABLE 4

| SEQ ID | | |
|---|---|---|
| NO | Name | Sequence |
| 19 | EpcamBind2 | DEVQHEVHELFVRIQELVVRGN |
| | | PEEARKLLEELEELAKKYNDPS |
| | | LHIAVEALERVIN |
| 20 | EpcamBind2_ | DEVQHEVHELMVRIHSLVVRGN |
| | cb10 | PEEARKLLEELEELAKKTNNPS |
| | (optimized) | YHIAVEALEHVIN |
| 21 | EpcamBind2_ | CgDEVQHEVHELMVRIHSLVVR |
| | cb10_cys | GNPEEARKLLEELEELAKKTNN |
| | | PSYHIAVEALEHVIN |

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:19-21. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:20.

In another embodiment, residues 1-20 relative to the reference sequence form a first alpha-helix, residues, residues 24-41 relative to the reference sequence form a second alpha-helix, and residues 44-57 relative to the reference sequence form a third alpha-helix. In a further embodiment, residues present in alpha-helices (“alpha-helical residues”) are identical to the reference sequence alpha-helical residues.

In a further aspect, the disclosure provides polypeptides that binds to human PDL1 receptor, wherein the polypeptide comprises the amino acid sequence of SEQ ID NO:3.

As disclosed in the examples that follow, the inventors have designed polypeptides according to SEQ ID NO:3 (exemplary such polypeptides are listed in Table 6), and conducted site saturation mutagenesis studies to determine permissible substitutions at each position, reflecting the allowable amino acids shown in Table 5. In some embodiments, these polypeptides bind to the extracellular portion of the human PDL1 receptor.

TABLE 5
SEQ ID NO: 3

| Residue number | Allowable amino acids |
|---|---|
| 1 Interface | P, D |
| 2 | C, G, V, L, S, N, Q, D, E, R, K, H |
| 3 | G, A, V, M, W, D, E, R, K |
| 4 Interface | G, V, I, M, L, T |
| 5 Interface | C, Y, W, T, N, D, R, K, H |
| 6 | I, M, T, N, R, K, H |
| 7 | C, V, M, F |
| 8 Interface | V, M, F, W |
| 9 | A, I, M, Y, W, T, N, Q, E, K, H |
| 10 | G, I, M, L, Y, R, K, H |
| 11 Interface | G, I, L, W |
| 12 Interface | C, G, A, S, N, Q, E, R, K, H |
| 13 | C, G, A, V, I, M, L, Y, W, N, Q, D, E, R, K, H |
| 14 | A, V, Y, F |
| 15 Interface | A, M, F, Y, W, S, N |
| 16 Interface | C, G, V, I, M, F, Y, S, N, E, R, K, H |
| 17 | C, A, I, M, L, W, S, N, Q, R, K, H |
| 18 | A, L, T, R |
| 19 Interface | G, A, V, I, M, L, Y, W, N, Q, D, E, R, K |
| 20 | G, A, V, M, F, S, T, Q, E, R, K, H |
| 21 | P, G, A, M, Y, E, R |
| 22 | C, I, M, L, F, W, S, T, N, Q, D, E, K, H |
| 23 | P, G, M, L, F, N, R, K, H |
| 24 | C, P, G, A, I, L, F, W, S, T, N, Q, D, E, K, H |
| 25 | A, M, F, S, N, R, K, H |
| 26 | G, A, V, F, S, T, N, R, H |
| 27 | C, G, A, V, I, M, L, F, Y, W, S, T, N, Q, R |
| 28 | C, V, I, F, Y, W, S, N, Q, D, E, H |
| 29 | C, A, V, K |
| 30 | L |
| 31 | A, I, M, W, S, T, N, D, E, R |
| 32 | G, A, V, I, L, S, D, E, R |
| 33 | A |
| 34 | C, G, A, V, L, F, Y, S, N, Q, E, R, H |
| 35 | G, A, I, M, L, F, W, T, Q, D, E, R |
| 36 | C, V, L, F, W, Q, D, R, K, H |
| 37 | A |
| 38 | C, G, V, I, F, Q, R, K, H |
| 39 | C, A, M, Y, W, N, Q, D, E, R |
| 40 | A, L, F, N, H |
| 41 | G, A, M, L, F, S, T, N, Q, D, R, K |
| 42 Interface | Q, D |
| 43 Interface | C, P, G, A, I, M, L, F, W, S, T, N, E, R, K, H |
| 44 Interface | G, F, S, T |
| 45 | L |
| 46 | A, I, T, E, R |
| 47 Interface | A, R, S |
| 48 Interface | V, F |
| 49 | A, V |
| 50 | I, L, F, T, N, Q, D, H |
| 51 Interface | A, V, I, L, W, T, D |
| 52 Interface | V, I, T, H |
| 53 | A, I, M, L, F, Y, W, S, T, N, Q, D, E, R, K, H |
| 54 Interface | P, T, N, Q, R |
| 55 Interface | P, V, L, W, N |
| 56 Interface | A, L, Y, W, R, K |

Table 5 also shows the position of interface residues in SEQ ID NO:3, at residues 1, 4, 5, 8, 11, 12, 15, 16, 19, 42, 43, 44, 47, 48, 51, 52, 54, 55, and 56. In one embodiment, interface residues 1, 4, 5, 8, 11, 12, 15, 16, 19, 42, 43, 44, 47, 48, 51, 52, 54, 55, and 56 are selected from the corresponding interface residues present in any one of SEQ ID NO:22-30 (sequences shown in Table 6).

The residue number of interface residues are listed in SEQ ID NO:3/Table 5, and the residue numbers of SEQ ID NO:22-30 in Table 6 are the same as those listed in SEQ ID NO:3/Table 5, except for:
SEQ ID NO:24 (PDL1_nterm-cys): This embodiment has two additional amino acid residues at the N-terminus relative to SEQ ID NO: 3; and
SEQ ID NO:30 (PDL1_dimer mer): This embodiment includes two copies of the PDL1 receptor binder. The first copy follows the residue numbering in SEQ ID NO:3/Table 5. The second copy of the sequence (i.e.: residue 1 of the second copy) begins after the lower case “ggsggs” (SEQ ID NO: 31) linker sequence.

TABLE 6

| SEQ | | |
|---|---|---|
| ID | | |
| NO | Name | Sequence |
| 22 | PPD11Bind | DEDTRRVMELLHEASELREEGDPERARE |
| | | VLEEAERLAKELGDPSLERFVQAHKRVY |
| 23 | PDL1Bind_ | DEDTDRVFELLNEFNELREEGDPERARE |
| | cb6 | VLEEAERLAKELSDPTLESFVQAIKRVY |
| | (Optimized) | |
| 24 | PDL1_nterm- | CgDEDTDRVFELLNEFNELREEGDPERA |
| | cys | REVLEEAERLAKELSDPTLESFVQAIKR |
| | | VY |
| 25 | PDL1_L10C | DEDTDRVFECLNEFNELREEGDPERARE |
| | | VLEEAERLAKELSDPTLESFVQAIKRVY |
| 26 | PDL1_E13C | DEDTDRVFELLNCFNELREEGDPERARE |
| | | VLEEAERLAKELSDPTLESFVQAIKRVY |
| 27 | PDL1_V29C | DEDTDRVFELLNEFNELREEGDPERARE |
| | | CLEEAERLAKELSDPTLESFVQAIKRVY |
| 28 | PDL1_E31C | DEDTDRVFELLNEFNELREEGDPERARE |
| | | VLCEAERLAKELSDPTLESFVQAIKRVY |
| 29 | PDL1_E39C | DEDTDRVFELLNEFNELREEGDPERARE |
| | | VLEEAERLAKCLSDPTLESFVQAIKRVY |
| 30 | PDL1_dimer | DEDTDRVFELLNEFNELREEGDPERARE |
| | | VLEEAERLAKELSDPTLESFVQAIKRVY |
| | | ggsggsDEDTDRVFELLNEFNELREEGD |
| | | PERAREVLEEAERLAKELSDPTLESFVQ |
| | | AIKRVY |

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:22-30. In one embodiment, interface residues 1, 4, 5, 8, 11, 12, 15, 16, 19, 42, 43, 44, 47, 48, 51, 52, 54, 55, and 56 are selected from the corresponding interface residues present in any one of SEQ ID NO:22-30.

In another embodiment, residues 1-20 relative to the reference sequence form a first alpha-helix, residues, residues 24-41 relative to the reference sequence form a second alpha-helix, and residues 44-55 relative to the reference sequence form a third alpha-helix. In a further embodiment, residues present in alpha-helices (“alpha-helical residues”) are identical to the reference sequence alpha-helical residues.

In one embodiment of any of the polypeptides of the disclosure, the polypeptide bind to their targets with nanomolar affinity as measured by octet. In the octet experiments, the target protein is immobilized onto an octet sensor and the kinetics of the binder binding to the target are measured over a range of binder concentrations to determine the binders affinity.

In one embodiment of any of the polypeptides of the disclosure, the polypeptides may comprise additional amino acid residues at the N- and/or C-terminus. In these embodiments, residue numbering are based on the references sequences as disclosed above, and percent identity requirements do not include any additional residues added at either termini. The additional residues may be any residues as deemed appropriate for an intended use. In some embodiments, the additional amino acid residues comprise a functional domain. In various non-limiting embodiments, the additional residues/functional domains may include, but are not limited to, detectable residues/domains (i.e.: GFP, etc.), expression tags (i.e.: N-terminal methionine or other residues), cysteine residues (for example, to facilitate covalent linkage to other moieties), functional domains (i.e.: cell or tissue targeting moieties; therapeutic peptide domains, diagnostic peptide domains, cytotoxins, etc.), additional copies of the EpCAM or PDL1 receptor binder (i.e.: multimers, such as dimers, trimers, etc., optionally linked by amino acid linkers, including but not limited to GS-rich linkers), scaffold linkages, etc.

As disclosed in the examples that follow, multimerization (such as dimerization) significantly increase binding affinity of the polypeptides for their targets. Thus, in another embodiment of any of the polypeptides of the disclosure, fusion proteins are provided comprising 2, 3, 4, or more polypeptides according to any embodiment or combination of embodiments herein, wherein the polypeptides may be directly linked, or may be linked via an amino acid linker. In one embodiment, the fusion proteins comprise a dimer of any of the polypeptides disclosed herein, directly linked, or linked via an amino acid linker.

In a further aspect, the present disclosure provides nucleic acids, including isolated nucleic acids, encoding the polypeptides and fusion proteins of the present disclosure. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the invention operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit.

In a further aspect, the present disclosure provides host cells that comprise the polypeptide, fusion protein, nucleic acid, and/or expression vector of any embodiment disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (RI. Freshney. 1987. Liss, Inc. New York, NY)). A method of producing a polypeptide or fusion protein according to the invention is an additional part of the invention. The method comprises the steps of (a) culturing a host according to this aspect of the invention under conditions conducive to the expression of the polypeptide, and (b) optionally, recovering the expressed polypeptide.

In another aspect, the present disclosure provides pharmaceutical compositions, comprising the polypeptide, fusion protein, nucleic acid, expression vector, or host cell of any embodiment or combination of embodiments herein and a pharmaceutically acceptable carrier. The pharmaceutical compositions of the disclosure can be used, for example, in the methods of the disclosure described herein. The pharmaceutical composition may further comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, fusion protein, nucleic acid, expression vector, or cell of any embodiment or combination of embodiments herein may be the sole active agent in the pharmaceutical composition, or the composition may further comprise one or more other active agents suitable for an intended use.

In another aspect, the disclosure provides methods for treating or limited development of cancer, autoimmune disease, or inflammation, comprising administering to a subject in need thereof an amount effective of the polypeptide, fusion protein, nucleic acid, expression vector, host cell, or pharmaceutical composition of any preceding claim to treat or limit development of the disorder. By way of non-limiting example, the PDL1 binders can act as checkpoint inhibitors capable of activating the immune system to attack cancer since they bind the same site as on PDL1 as antibody checkpoint inhibitors.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The subject may be any subject that has a relevant disorder. In one embodiment, the subject is a mammal, including but not limited to humans, dogs, cats, horses, cattle, etc. In one embodiment, the subject is a human subject.

### EXAMPLES

We developed mini-protein binders capable of targeting either the human epithelial cell adhesion molecule (EpCAM) receptor or human programmed death-ligand 1 (PDL1) receptor. These receptors are important targets for a number of human diseases such as but not limited to cancer, autoimmunity, and inflammation. The de novo designed proteins bind to their targets with low to mid nanomolar affinities as determined by octet. Additionally site saturation mutagenesis (SSM) of the binders displayed on yeast was conducted to reveal the functional tolerance of all possible single point mutations. SEC shows the designs are well behaved monodisperse proteins, and cell binding assays show the designs can target human cells expressing their respective target receptors.

### Introduction and Results

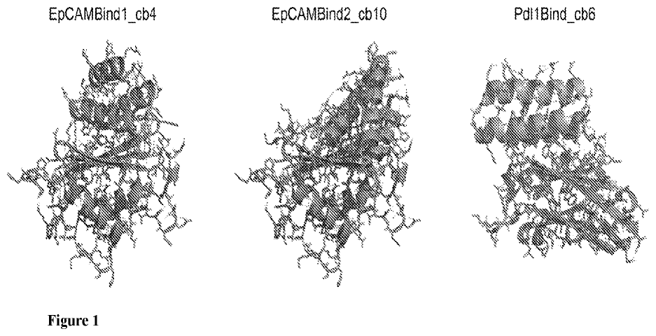

Human EpCAM and PDL1 receptors are implicated in a range of diseases such as cancer, autoimmunity and inflammation. In order to generate binders capable of targeting these receptors, which could have clinical applications, we designed de novo miniproteins following a published protocol involving rifdock and Rosetta™ sequence design. Designs were first screened for binding their respective target proteins by yeast surface display, and the best binders (EpcamBind1, EpcamBind2, and Pdl1Bind) were further optimized by site saturation mutagenesis (SSM) and subsequent combinatorial libraries based on the best single point mutations. This led to the generation of optimized EpcamBind1_cb4, EpcamBind2_cb10, which bind the extracellular portion of human EpCAM receptor; and Pdl1Bind_cb6 which binds the extracellular portion of human PDL1 (see sequences Tables and FIG. 1).

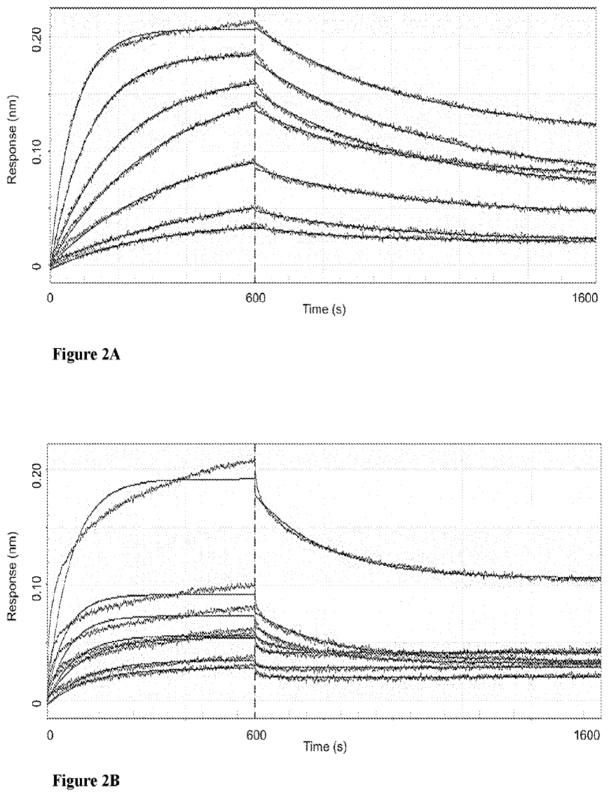

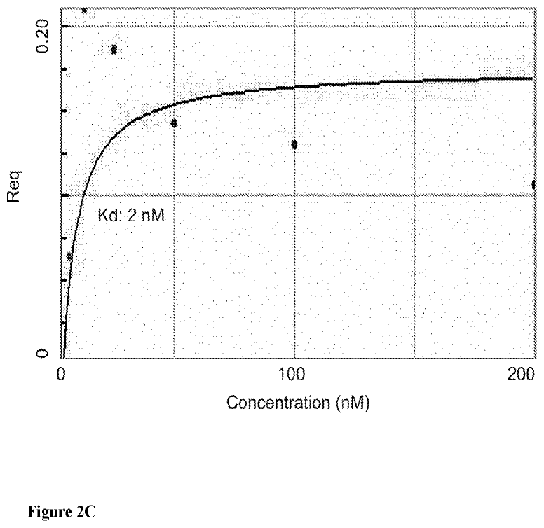

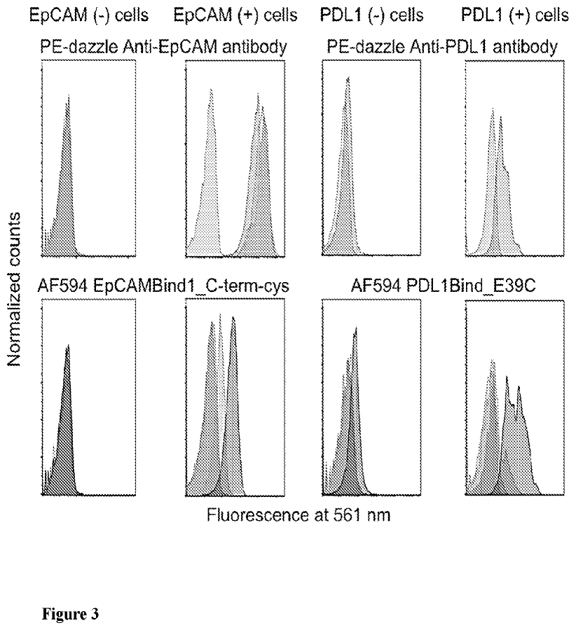

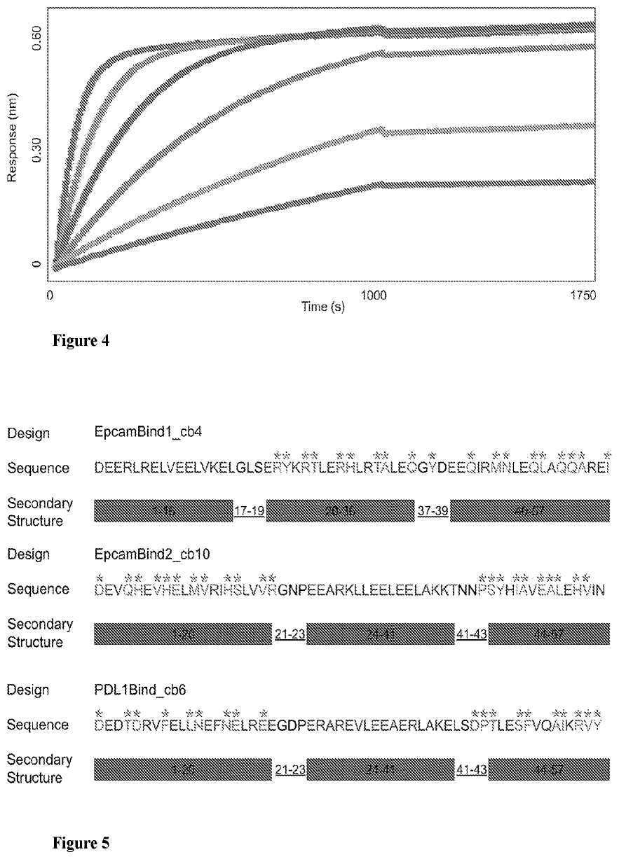

These proteins bind their targets with low to mid nanomolar affinities based on octet data (see FIG. 2A-C). We subsequently identified positions on the designs based on the SSM data where we could introduce a surface cysteine residue to enable conjugation of small molecules of interest to the proteins, which may facilitate cell labeling and drug targeting applications. We showed that these variants labeled with Alexa Fluor 594 (AF594) are capable of specifically binding cells expressing their respective targets (see FIG. 3) and that we can significantly increase binding affinity of the EpCAMBind1 to low picomolar via creation of a gs linked fusion dimer (see FIG. 4). We further show that the introduction of AF594 does not impact binding or stability of the proteins by circular dichroism, except for PDL1_L10C which seems poorly folded with and without the label. The rest of the designs with and without conjugated AF594 are well behaved, remain folded to 60-70° C., and completely refold upon cooling to room temperature. We show sequence, secondary structure information, and interface positions for EpcamBind1_cb4, EpcamBind2_cb10, and, PDL1Bind_cb6 in FIG. 5.

The EpCAM binders and PDL1 binders have been conjugated to the cytotoxin MMAE through site specific cysteines and shown potent in vitro toxicity to cancer cell lines expressing their respective target proteins (data not shown).

1. A polypeptide that
(a) binds to human epithelial cell adhesion molecule (EpCAM) receptor, wherein the polypeptide comprises the amino acid sequence of SEQ ID NO:1; or
(b) binds to human EpCAM receptor, wherein the polypeptide comprises the amino acid sequence of SEQ ID NO:2; or
(c) binds to human programmed death-ligand 1 (PDL1) receptor, wherein the polypeptide comprises the amino acid sequence of SEQ ID NO:3.

2. The polypeptide of claim 1, wherein interface residues 21, 22, 24, 25, 28, 29, 32, 33, 36, 42, 45, 46, 49, 50, 52, 53, 54, and 57 relative to SEQ ID NO:1 are selected from the corresponding interface residues present in any one of SEQ ID NO:4-18.

3. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:4-18.

4.-8. (canceled)

9. The polypeptide of claim 19, wherein interface residues 1, 4, 5, 7, 8, 9, 11, 12, 15, 16, 19, 20, 43, 44, 45, 47, 48, 50, 51, 52, 54, and 55 relative to SEQ ID NO:2 are selected from the interface residues present in the amino acid sequence selected from SEQ ID NO:19-21.

10. The polypeptide of claim 9, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:19-21.

11.-12. (canceled)

13. The polypeptide of claim 1, wherein residues 1-20 relative to SEQ ID NO:2 form a first alpha-helix, residues 24-41 relative to SEQ ID NO:2 form a second alpha-helix, and residues 44-57 relative to SEQ ID NO:2 form a third alpha-helix.

14. The polypeptide of claim 13, wherein alpha-helical residues are identical to the reference sequence alpha-helical residues.

15. (canceled)

16. The polypeptide of claim 1, wherein interface residues 1, 4, 5, 8, 11, 12, 15, 16, 19, 42, 43, 44, 47, 48, 51, 52, 54, 55, and 56 relative to SEQ ID NO:3 are selected from the interface residues present in the amino acid sequences selected from SEQ ID NO:22-30.

17. The polypeptide of claim 16, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:22-30.

18.-19. (canceled)

20. The polypeptide of claim 16, wherein residues 1-20 relative to SEQ ID NO:3 form a first alpha-helix, residues, residues 24-41 relative to SEQ ID NO:3 form a second alpha-helix, and residues 44-55 relative to SEQ ID NO:3 form a third alpha-helix.

21.-22. (canceled)

23. A fusion protein comprising 2, 3, 4, or more polypeptides according to claim 1, wherein the polypeptides may be directly linked, or may be linked via an amino acid linker.

24. A nucleic acid encoding the polypeptide of claim 1.

25. An expression vector comprising the nucleic acid of claim 24 operatively linker to a suitable regulatory control element.

26. A host cell comprising the expression vector of claim 25.

27. A pharmaceutical composition, comprising:
(a) the polypeptide of claim 1; and
(b) a pharmaceutically acceptable carrier.

28. (canceled)

29. A method for treating or limiting development of cancer, autoimmune disease, or inflammation, comprising administering to a subject in need thereof an amount effective of the polypeptide of claim 1 to treat or limit development of the disorder.
